(19) United States
(12) Patent Application Publication
(10) Pub. No.: US 2017/0231611 A1
(43) Pub. Date: Aug. 17, 2017

(54) SPECIMEN RETRIEVAL BAGS AND SPECIMEN RETRIEVAL SYSTEMS

(71) Applicant: Covidien LP, Manfield, MA (US)

(72) Inventors: Henry E. Holsten, Hamden, CT (US); Stanislaw Marczyk, Stratford, CT (US); Eugene A. Stellon, Burlington, CT (US); Kenneth W. Horton, Jr., South Glastonbury, CT (US); Terri Wells, North Branford, CT (US); Michael Tarnoff, Sharon, MA (US); Stanislaw Kostrzewski, Newtown, CT (US); Jaroslaw T. Malkowski, Trumbull, CT (US); Anibal Rodrigues, Jr., Emeryville, CA (US)

(21) Appl. No.: 15/504,480

(22) PCT Filed: Aug. 19, 2014
(86) PCT No.: PCT/US2015/041465

§ 371 Date: Feb. 16, 2017

Related U.S. Application Data
(60) Provisional application No. 62/039,002, filed on Aug. 19, 2014.
(60) Provisional application No. 62/039,003, filed on Aug. 19, 2014.
(60) Provisional application No. 62/039,023, filed on Aug. 19, 2014.
(60) Provisional application No. 62/039,037, filed on Aug. 19, 2014.
(60) Provisional application No. 62/038,999, filed on Aug. 19, 2014.
(60) Provisional application No. 62/038,996, filed on Aug. 19, 2014.
(60) Provisional application No. 62/038,994, filed on Aug. 19, 2014.
(60) Provisional application No. 62/038,980, filed on Aug. 19, 2014.
(60) Provisional application No. 62/038,982, filed on Aug. 19, 2014.
(60) Provisional application No. 62/038,977, filed on Aug. 19, 2014.
(60) Provisional application No. 62/038,973, filed on Aug. 19, 2014.
(60) Provisional application No. 62/038,972, filed on Aug. 19, 2014.

(51) Int. Cl. A61B 17/00; A61B 1/00; A61B 1/313

(52) U.S. Cl. CPC A61B 17/00234; A61B 1/3132; A61B 1/00154; A61B 2017/00287; A61B 2017/0034; A61B 2017/320024

(57) ABSTRACT

A specimen bag configured for viewing the contents therein during a surgical procedure is provided. The specimen bag includes an open end and a closed end and defines a cavity. An appendage extends from the bag and defines a channel in communication with the cavity of the bag for receiving a surgical instrument.

### CROSS-REFERENCE TO RELATED APPLICATION

The present application claims the benefit of and priority to U.S. Provisional Patent Application Ser. No. 62/038,973, filed Aug. 19, 2014; U.S. Provisional Patent Application Ser. No. 62/038,972, filed Aug. 19, 2014; U.S. Provisional Patent Application Ser. No. 62/038,977, filed Aug. 19, 2014; U.S. Provisional Patent Application Ser. No. 62/038,980, filed Aug. 19, 2014; U.S. Provisional Patent Application Ser. No. 62/038,982, filed Aug. 19, 2014; U.S. Provisional Patent Application Ser. No. 62/038,994, filed Aug. 19, 2014; U.S. Provisional Patent Application Ser. No. 62/038,996, filed Aug. 19, 2014; U.S. Provisional Patent Application Ser. No. 62/038,999, filed Aug. 19, 2014; U.S. Provisional Patent Application Ser. No. 62/039,002, filed Aug. 19, 2014; U.S. Provisional Patent Application Ser. No. 62/039,003, filed Aug. 19, 2014; U.S. Provisional Patent Application Ser. No. 62/039,023, filed Aug. 19, 2014; and U.S. Provisional Patent Application Ser. No. 62/039,037, filed Aug. 19, 2014. The contents of each of these applications are incorporated by reference herein in their entirety.

### BACKGROUND

Technical Field

The present disclosure relates generally to tissue removal during minimally invasive surgical procedures using specimen bags. More particularly, the present disclosure relates to specimen bags and/or methods of using specimen bags for viewing tissue within the specimen bags within a body cavity.

Background of Related Art

During a minimally invasive surgical procedure, such as, for example, a laparoscopic cholecystectomy, following placement of the gallbladder or other tissue to be removed within a specimen bag, an open end of the specimen bag is retracted through an incision in the abdominal wall to permit access to the interior of the specimen bag. A morcellator may then be received within the specimen bag to morcellate and, in most instances, remove the tissue. Once the tissue is removed from the specimen bag, or once the tissue has been sufficiently reduced in size to permit removal of the specimen bag through the incision, the specimen bag is removed through the incision. The ability to visualize the contents of the specimen bag during morcellation would enable a surgeon to direct the tissue into the morcellator and/or enable a surgeon to determine when the tissue has been sufficiently morcellated thereby permitting removal of the specimen bag through the incision.

It would, therefore, be advantageous to provide specimen bags and methods of using the specimen bags that enable a surgeon to view the contents of the specimen bags during a laparoscopic procedure within a body cavity.

### SUMMARY

Accordingly, a specimen bag configured for viewing the contents therein during a surgical procedure is provided. The specimen bag includes a bag having an open end and a closed end and defining a cavity, and an appendage extending from the bag and defining a channel in communication with the cavity of the bag.

In embodiments, a proximal end of the appendage is sealed. The specimen bag may further include at least one additional appendage extending from the bag. The appendage may be configured to receive a surgical instrument through the channel in a sealed manner. The appendage may include a seal member for sealing the channel in the absence of a surgical instrument received therethrough. The appendage may include a length sufficient to be received through an opening in tissue when the bag is disposed within in a body cavity.

Also provided is a method of viewing the contents of a specimen bag. The method including placing tissue within a cavity of a specimen bag, opening a proximal end of an appendage extending from the specimen bag, and receiving an instrument through the appendage into the cavity of the specimen bag.

In embodiments, the method further includes directing the proximal end of the appendage through an incision in tissue prior to opening the proximal end. The method may further include sealing the appendage after removing the instrument from the appendage. In addition, the method may include manipulating the tissue within the cavity. The method may also include receiving an open end of the specimen bag through an opening in tissue.

Another specimen bag configured for viewing the contents therein during a surgical procedure is provided. The specimen bag includes a bag having an open end and a closed end and defining a cavity, and a visualization member for viewing within the cavity of the bag.

In embodiments, the visualization member includes a transparent window formed in the bag. The visualization member may instead include a viewing port secured to the bag. The specimen bag may include at least one additional visualization member.

Also provided is another method of viewing the contents of a specimen bag. The method includes placing tissue within a cavity of a specimen bag including a visualization member, and positioning a scope adjacent the visualization member of the specimen bag. The method may further include receiving an open end of the specimen bag through an opening in tissue.

In addition, a specimen bag configured for viewing the contents therein during a surgical procedure is provided. The specimen bag includes a bag having an open end and a closed end and defining a cavity, and a port extending through a wall of the bag, the port defining a resealable opening for selectively receiving a surgical instrument.

In embodiments, the specimen bag further includes at least one addition port extending through the wall of the bag.

A further method of viewing the contents of a specimen bag is provided. The method includes placing tissue within a cavity of a specimen bag through an open end of the specimen bag, and receiving a scope through a resealable opening in a port extending through a wall of the bag.

A specimen bag configured for viewing the contents therein during a surgical procedure is provided. The specimen bag includes a bag having an open end and a closed end and defining a cavity, and a divider for separating the cavity into a first chamber for receiving tissue and a second chamber for receiving a scope. The divider may be formed of a transparent material.

Also provided is a method of viewing the contents of a specimen bag. The method includes placing tissue within a first chamber of a bag, receiving a scope within a second chamber of the specimen bag, and directing the scope to view through a transparent divider separating the first chamber and the second chamber.

A specimen retrieval system is also provided. The specimen retrieval system includes a specimen bag having an open end and a closed end and defining a cavity, and a wireless camera disposed within the cavity of the bag specimen. The specimen retrieval system may also include a steering mechanism having a distal end configured for receipt within the cavity of the specimen bag. The wireless camera may be secured to a distal end of the steering mechanism.

Another method of viewing the contents of a specimen bag is provided. The method includes placing tissue within a specimen bag, receiving a wireless camera within a cavity of the specimen bag, and positioning the wireless camera for viewing within the cavity of the specimen bag.

Still another specimen retrieval system is provided. The specimen retrieval system includes a specimen bag having an open end and a closed end and defining a cavity, and a first appendage including an expandable flange for fixedly securing the appendage to the specimen bag.

Still another method of viewing the contents of a specimen bag is provided. The method includes placing tissue within a specimen bag, receiving a collapsible flange of an appendage through a wall of the specimen bag when the collapsible flange is in a collapse condition, expanding the flange of the appendage, and engaging the collapsible flange with the wall of the specimen bag.

Another specimen bag configured for viewing the contents therein during a surgical procedure is provided. The specimen bag includes a bag having an open end and a closed end and defining a cavity, and a first appendage extending from the bag and defining a channel in communication with the cavity of the bag. The first appendage may include a pointed distal end for piercing tissue.

Yet another method of viewing the contents of a specimen bag is provided. The method includes placing tissue within a cavity of a specimen bag having a first appendage extending therefrom, and piercing tissue using a sharpened distal end of the appendage.

Still yet another specimen bag configured for viewing the contents therein during a surgical procedure is provided. The specimen bag includes an outer bag and an inner bag received within the outer bag. The inner bag may be formed of a translucent material for viewing the contents thereof.

Still yet another method of viewing the contents of a specimen bag is provided. The method includes placing tissue within an inner bag of a specimen bag, insufflating an outer bag of the specimen bag, forming an opening through the outer bag of the specimen bag, and receiving a scope through the outer bag for viewing of the tissue within the inner bag.

Another specimen retrieval system is provided. The specimen retrieval system includes a specimen bag having an open end and a closed end and defining a cavity, and a thermal imaging device for viewing the contents of the specimen bag.

Another method of viewing the contents of a specimen bag is provided. The method includes placing tissue within a specimen bag, and positioning a thermal imaging device adjacent the specimen bag to provide a thermal image of the tissue within the specimen bag

Yet another specimen retrieval system is provided. The specimen retrieval system includes a specimen bag having an open end and a closed end and defining a cavity, and an ultrasound device for viewing the contents of the specimen bag.

Yet another method of viewing the contents of a specimen bag is provided. The method includes placing tissue within the specimen bag, and positioning an ultrasound device in proximity to the specimen bag external a body cavity while the specimen bag is in the body cavity.

Another specimen retrieval system is provided. The specimen retrieval system including a specimen bag having an open end and a closed end and defining a cavity, a fiber optic scope receivable with the cavity of the specimen bag through the open end, and a morcellator for morcellating tissue within the cavity of the specimen bag receivable through the open end of the specimen bag simultaneously with the fiber optic scope.

Another method of viewing the contents of a specimen bag is provided. The method including placing tissue in a cavity of a specimen bag through an open end of the specimen bag, receiving a morcellator through the open end of the specimen bag, and receiving a fiber optic scope through the open end of the specimen simultaneously with the morcellator.

Yet another specimen retrieval system is provided. The specimen retrieval system including a specimen bag having an open end and a closed end and defining a cavity, and a multi-lumen access port selectively receivable within the open end of the specimen bag.

Yet another method of viewing the contents of a specimen bag. The method including placing a specimen bag within the body cavity of a patient through an incision, receiving tissue in a cavity of a specimen bag through an open end of the specimen bag, withdrawing the open end of the specimen bag through the incision, and receiving a multi-lumen access port within the open end of the specimen bag, and receiving a scope through a lumen of the multi-lumen access port.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective side view of a specimen bag according to an embodiment of the present disclosure;

FIG. 1A is cross-sectional top view taken along line 1A-1A of FIG. 1;

FIG. 2 is a perspective side view of a specimen bag according to another embodiment of the present disclosure;

FIG. 3 is a perspective side view of a specimen bag according to another embodiment of the present disclosure;

FIG. 3A is an enlarged perspective side view of a viewing port of the specimen bag shown in FIG. 3;

FIG. 4 is a perspective side view of a specimen bag according to another embodiment of the present disclosure;

FIG. 5 is a perspective side view of a specimen bag according to another embodiment of the present disclosure;

FIG. 5A is a perspective side view of a steering mechanism suitable for use with the specimen bag of FIG. 5, in a first position;

FIG. 5B is a perspective side view of the steering mechanism shown in FIG. 5A, in a second position;

FIG. 6 is a perspective side view of a specimen bag according to another embodiment of the present disclosure;

FIG. 7 is a perspective side view of a specimen bag according to an embodiment of the present disclosure;

FIG. 8 is a perspective side view of a specimen bag according to an embodiment of the present disclosure;

FIG. 9 is a perspective side view of a specimen bag according to an embodiment of the present disclosure;

FIG. 10 is a perspective side view of a specimen bag according to an embodiment of the present disclosure;

FIG. 11 is a perspective side view of a specimen bag according to an embodiment of the present disclosure; and

FIG. 12 is a perspective side view of a specimen bag according to an embodiment of the present disclosure.

### DETAILED DESCRIPTION

Embodiments of the present disclosure will now be described in detail with reference to the drawings, in which like reference numerals designate identical or corresponding elements in each of the several views. As used herein, the term distal refers to the portion of the instrument which is farthest from the user, while the term proximal refers to that portion of the instrument which is closest to the user. In the following description, well-known functions or constructions are not described in detail to avoid obscuring the present disclosure in unnecessary detail.

As used herein with reference to the present disclosure, the terms laparoscopic and endoscopic are interchangeable and refer to instruments having a relatively narrow operating portion for insertion into a cannula or a small incision in the skin. Laparoscopic and endoscopic also refer to minimally invasive surgical procedures. It is believed that the present disclosure may find use in any procedure where access to the interior of the body is limited to one or more relatively small incisions, with or without the use of a cannula or other access port, as in minimally invasive procedures.

Various specimen bags, instruments, and methods for inserting and retrieving the specimen bags from within a patient are known. For example, commonly owned U.S. Pat. Nos. 5,647,372, 5,465,731, 6,409,733, 5,037,379, and 5,735,289, and U.S. Patent Application Publication No. 2014/0135788 disclose various specimen bags, applicators, and methods for deploying the specimen bags. The contents of these patents and publications are incorporated by reference herein in their entirety.

The aspects of the present disclosure may be modified for use with various methods for retrieving tissue during minimally invasive procedures. Although the embodiments of the present disclosure will be described with reference to a cholecystectomy, e.g., gallbladder removal, the embodiments of the present disclosure may be used or modified for use with other minimally invasive procedures, e.g., appendectomies, nephrectomies, colectomy, splenectomy. Unless otherwise noted, the specimen bags of the present disclosure are formed of rip stop nylon or other suitable material. The specimen bags of the present disclosure may be closed using a drawstring or in any other suitable manner, and may include any feature necessary for deploying and/or retrieving the specimen bag from within a body cavity.

As described above, during a minimally invasive procedure, once the tissue to be removed is received within a specimen bag, an open end of the specimen bag is withdrawn through an incision, either directly or through a cannula and/or an access port, to permit the introduction of a morcellator within the specimen bag while the portion of the specimen bag containing tissue remains in the body cavity. As used herein, the term morcellator refers to a surgical instrument for cutting, mincing up, liquefying, or morcellating, tissue into smaller pieces. Morcellators may be powered or hand-operated, and are generally configured to extract the tissue from the specimen bag, via, e.g., a vacuum tube or through the operation of the cutting mechanism, as the tissue is morcellated. The empty specimen bag is then withdrawn from the patient through the incision, either directly or through the cannula and/or the access port. Alternatively, the morcellated tissue can remain within the specimen bag and be removed from the patient through the incision along with the specimen bag.

As used herein, the term scope refers to any instrument capable of transmitting information, such as an image to a display, e.g., a monitor, for observation by a surgeon. The scope may have a fixed viewing end or the viewing end may be articulable. The scope may have various lenses, including, for example, panoramic, zoom, or fixed. The scope may be configured for access into a body cavity through an incision, either directly or through a cannula and/or an access port. The scope may include a traditional camera, fiber optic camera, and/or night vision.

The morcellator and the scope in combination with the specimen bag and various other instruments, including, but limited to, trocars, cannulas, access ports, and graspers, form systems for removing tissue from a body cavity during minimally invasive surgery. It is envisioned that the specimen bags of the present disclosure may be modified for use with various instruments. It is further envisioned that the methods of using the specimen bags of the present disclosure may be modified to accommodate needs of a given procedure and/or the preferences of the surgeon. It is further envisioned that the embodiments disclosed herein may be used to remove any tissue or object from the body. Examples provided herein, such as gallbladders, are merely exemplary and are not intended to limit the scope of the invention.

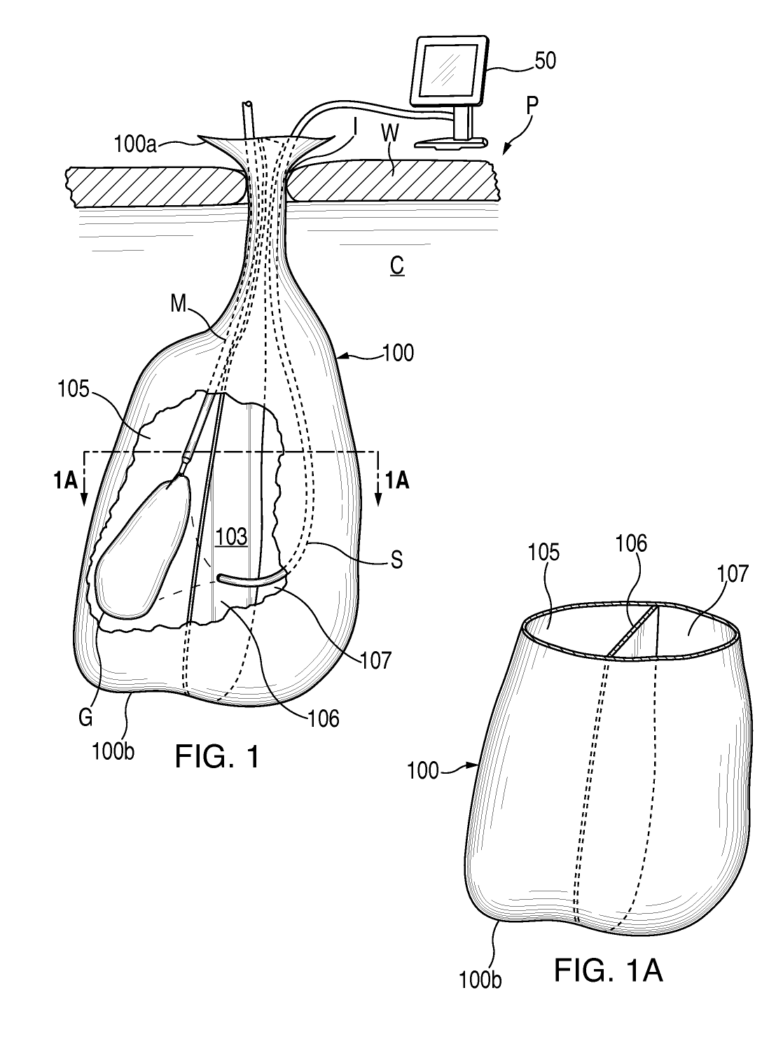

Referring initially to FIG. 1, an embodiment of the present disclosure is shown generally as specimen bag 100. The specimen bag 100 includes an open end 100a and a closed end 100b and defines a cavity 103 which is divided into a first chamber 105 and a second chamber 107 by a divider 106. Tissue to be removed from the patient, e.g., gallbladder “G”, is received in the first chamber 105. A scope “S” is received in the second chamber 107 and transmits an image to a display, e.g., monitor 50. The divider 106 is formed of a transparent material to permit viewing of the contents of the first chamber 105 from within the second chamber 107. The first chamber 105 and the second chamber 107 of specimen bag 100 are accessed through the open end 100a of the specimen bag 100 which is pulled through or positioned adjacent to the incision “I”.

During a laparoscopic procedure, the specimen bag 100 is received and positioned within the body cavity “C” of a patient using known methods. See, for example, the '372 patent, the content of which was previously incorporated by herein reference. The tissue to be removed from the patient, e.g., gallbladder “G”, is placed within the first chamber 105 of the specimen bag 100 through the open end 100a of the specimen bag 100. The open end 100a is then retracted through incision “I” of the patient “P”, to provide the surgeon with access to the cavity 103 of the specimen bag 100, and more particularly, to access the first chamber 105 and the second chamber 107. A morcellator “M” is then positioned within the first chamber 105 through the open end 100a of the specimen bag 100 and a scope “S” is positioned within the second chamber 107 through the open end 100a of the specimen bag 100.

Insufflation gas can be provided to the first and second chambers 105, 107, respectively, of the specimen bag 100 to enlarge the first and second chambers 105, 107 and improve visualization. In embodiments, insufflation gas is provided to the first and or second chambers 105, 107 of the specimen bag 100 directly through the morcellator or scope “S”. Alternatively, other means, e.g., an insufflation trocar, can be used to insufflate the first and second chambers 105, 107. As the morcellator “M” is operated within the first chamber 105, the contents of the first chamber 105 are viewed by the surgeon on a display, e.g., monitor 50, by directing the scope “S” within the second chamber 107 to view the first chamber 105 through the divider 106, at least a portion of which may be transparent or translucent. Upon removal of the morcellated tissue from the first chamber 105 or upon visual determination by the surgeon that the tissue has been sufficiently reduced in size to permit removal of the specimen bag 100 through the incision “I”, the morcellator “M” is removed from within the first chamber 105 and the scope “S” is removed from within the second chamber 107. The specimen bag 100 (and the morcellated tissue) is then removed from the body cavity through the incision “I” of the patient “P”.

It is envisioned that the first and second chambers may be positioned in any number of ways relative to each other. For example, as shown in FIG. 1, the first and second chambers 105, 107 are oriented generally parallel to the longitudinal access of the bag. Alternatively the first and second chambers may be oriented transverse to the bag, or at an angle. Additionally, the first and second chambers may be of generally equal size, or the first chamber may be larger or smaller than the second chamber. Additionally, there may be at least a third chamber and at least a second at least partially transparent or translucent wall or divider.

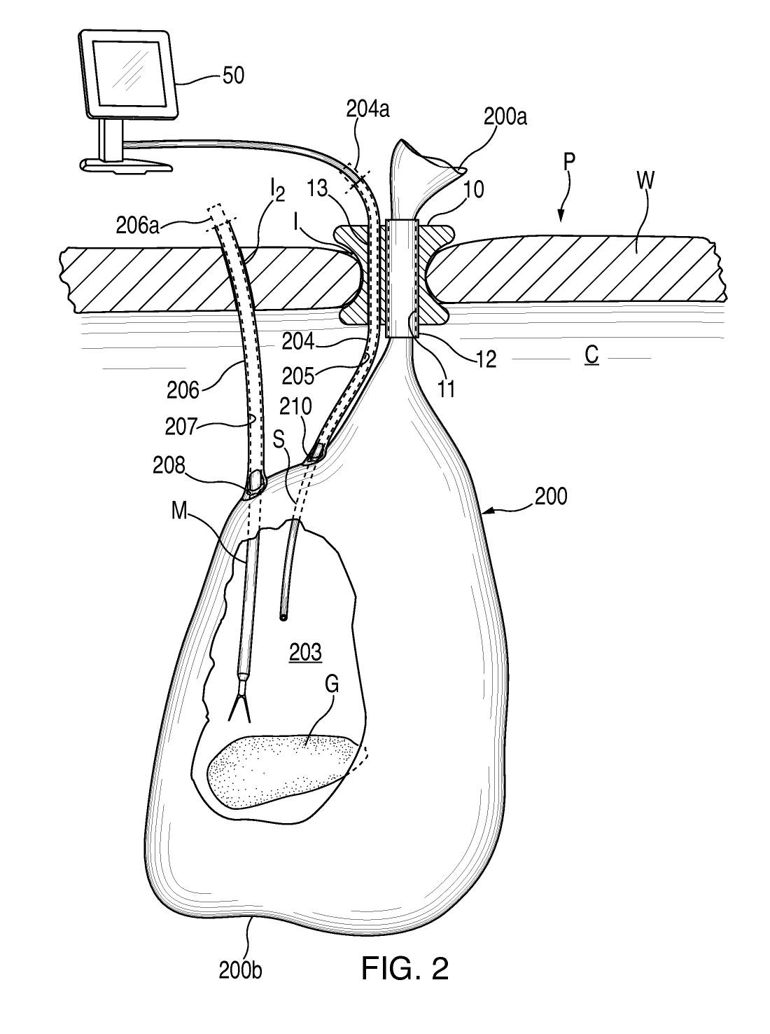

With reference now to FIG. 2, another embodiment of a specimen bag according to the present disclosure is shown generally as specimen bag 200. The specimen bag 200 includes an open end 200a and a closed end 200b and defines a cavity 203 for receiving tissue to be removed from the patient, e.g., gallbladder “G”. The first and second appendages 204, 206 are integrally formed with or securely affixed to the specimen bag 200. Although shown including two appendages, it is envisioned that the specimen bag 200 may include only a single appendage or more than two appendages. Each of the first and second appendages 204, 206 defines a channel 205, 207, respectively, communicating with and providing access to the cavity 203 of the specimen bag 200. The channels 205, 207 of the first and second appendages 204, 206 are dimensioned to selectively receive a morcellator “M”, a scope “S”, or other instrument (not shown) in a fluid tight manner.

In embodiments, the first and second appendages 204, 206 are configured to extend from the specimen bag 200 and are received through an incision in the abdominal wall. More particularly, the first and second appendages 204, 206 may be received through an incision “I” or through an opening 13 in an access port 10 received within the incision “I” in which the open end 200a of the specimen bag 200 is or will be received and/or through an alternate incision, e.g., second incision “I₂”. Alternatively, either or both of the first and second appendages 204, 206 are configured to remain within the body cavity “C” of the patient during a procedure. In this manner, the channels 205, 207 can be accessed with instruments received within the body cavity “C”, e.g., forceps, graspers. The first and second appendages 204, 206 each include a sealed end 204a, 206a, respectively, which can be unsealed, such as by detaching a cap (not shown), or cutting or puncturing the sealed ends 204a, 206a, during a procedure to permit access to the respective channels 205, 207 of the first and second appendages 204, 206, respectively.

Following the morcellation of the tissue with the specimen bag 200 and removal of the morcellator “M” and the scope “S” from within the channels 205, 207, respectively, of the respective first and second appendages 204, 206, the channels 205, 207 may be resealed in any suitable manner. For example, the channels 205, 207 may be sealed by welding, or by folding the ends of the first and second appendages 204, 206 and suturing, stapling or otherwise securing the folded ends. By sealing channels 205, 207 of respective first and second appendages 204, 206, any material remaining in the cavity 203 of the specimen bag 200 is prevented from leaking from the first and second appendages 204, 206 of the specimen bag 200 as the specimen bag 200 is removed from the patient.

During a laparoscopic procedure, the specimen bag 200 is received and positioned within the body cavity “C” of a patient using known methods. The tissue to be removed from the patient, e.g., gallbladder “G”, is placed within the cavity 203 of the specimen bag 200 through the open end 200a of the specimen bag 200. The open end 200a of the specimen bag 200 is then retracted through the incision “I” of the patient “P”.

At any time during the procedure, the surgeon may use forceps, graspers, and/or another instrument (not shown) to grasp and direct the first and second appendages 204, 206 through the incisions “I”, “I₂”. Alternatively, either or both of the first and second appendages 204, 206 may be accessed while remaining entirely within the body cavity “C”. The sealed ends 204a, 206a of the appendages 204, 206, respectively, are then unsealed, as described above, to provide access to the respective channels 205, 207 of the first and second appendages 204, 206, respectively. An anchor or other holding means (not shown) may be attached to each of the first and second appendages 204, 206 to prevent the first and second appendages 204, 206 from being retracted back within the body cavity “C”.

The scope “S” and the morcellator “M” are then inserted through the respective channels 205, 207 of the first and second appendages 204, 206, respectively, and guided into the cavity 203 of the specimen bag 200 to permit viewing and morcellation of the contents of the specimen bag 200. Alternatively, the scope “S” and/or the morcellator “M” is received through the open end 200a of the specimen bag 200. Insufflation gas can be provided to the cavity 203 of the specimen bag 200 through either or both of the scope “S” and the morcellator “M” or through an alternative means, e.g., an insufflation trocar, to expand the cavity 203 of the specimen bag 200 and to improve visualization. It is envisioned that insufflation gas may be provided to the cavity 203 through the open end 200a of the specimen bag 200, through either or both of the first and second appendages 204, 206, through a third appendage (not shown), and/or through an opening (not shown) created in the wall of the specimen bag 200.

The positioning of the scope “S” within the cavity 203 of the specimen bag 200 permits a surgeon to view the contents of the cavity 203 of the specimen bag 200 during operation of the morcellator “M”. Upon removal of the morcellated tissue from the cavity 203 of the specimen bag 200 or upon visual determination that the tissue has been sufficiently reduced in size to permit removal of the specimen bag through the incision “I”, the scope “S” and morcellator “M” are withdrawn from the cavity 203 of the specimen bag 200 and from the channels 205, 207 of the first and second appendages 204, 206, respectively.

The channels 205, 207 of the first and second appendages 204, 206, respectively, are then sealed to prevent leakage of any material from within the cavity 203 of the specimen bag 200 into the body cavity “C” during removal of the specimen bag 200 through incision “I”. As described above, the channels 205, 207 of the respective first and second appendages 204, 206 may be sealed by welding or by folding the ends of the first and second appendages 204, 206 and suturing, stapling or otherwise securing the folded ends of the first and second appendages 204, 206. The specimen bag 200 is then removed from the body cavity “C” through the incision “I”. Alternatively, graspers may be used to grasp the open ends of the first and second appendages 204, 206 and the first and second appendages 204, 206 can be used to withdraw the specimen bag 200 from within the body cavity “C” of the patient “P”.

The first and second appendages 204, 206 may include a seal 208, 210, for example at the juncture of the first and second appendages 204, 206, respectively, and the specimen bag 200, at the tip of the first and second appendages 204, 206, or along the length of the first and second appendages for maintaining a seal in the absence of an object and/or about an object inserted through the first and second appendages 204, 206. The seals 208, 210 may be any conventionally known seal or valve, such as a duckbill, joker, or zero-closure. Alternatively, seal 208, 210 may include a combination of seals.

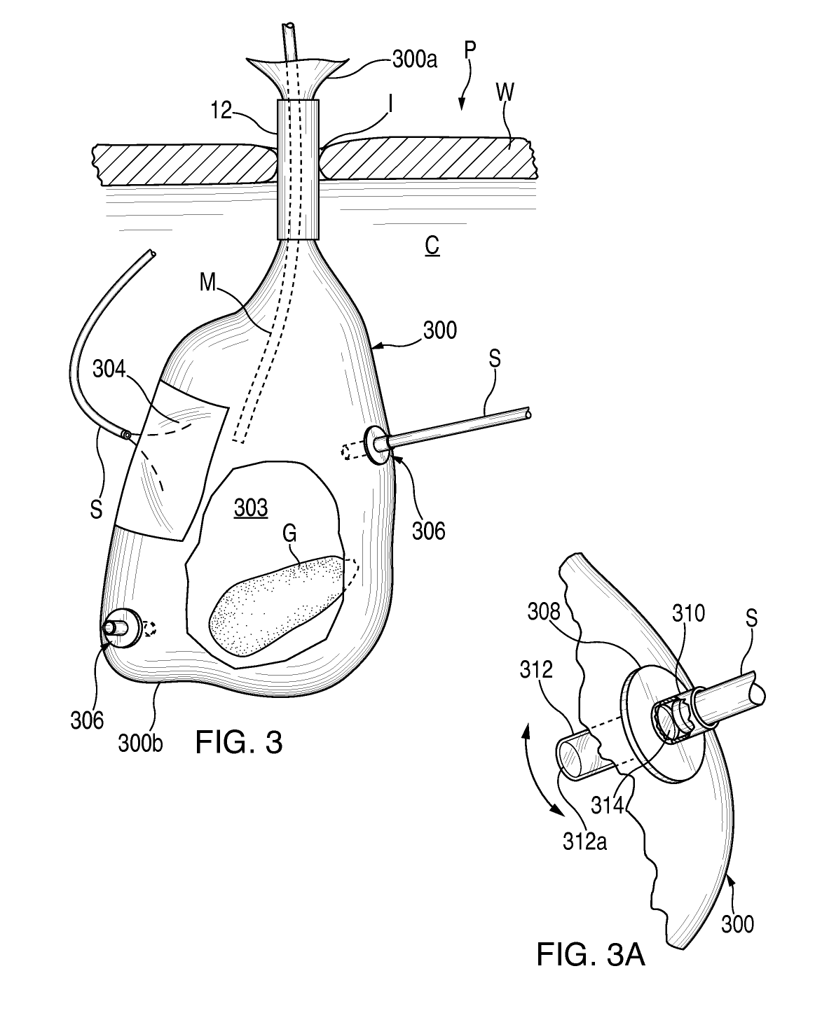

With reference now to FIG. 3, another embodiment of a specimen bag according to the present disclosure is shown generally as specimen bag 300. The specimen bag 300 includes an open end 300a and a closed end 300b and defines a cavity 303 for receiving tissue to be removed from the patient, e.g., gallbladder “G”. The specimen bag 300 further includes one or more windows 304 and/or one or more viewing ports 306 to permit viewing of the contents of the specimen bag 300.

The window 304 may be integrally formed with or securely affixed, for example by welding or molding, to a wall of the specimen bag 300 and is formed of a transparent or translucent material that permits viewing of at least some of the contents of the specimen bag 300 from outside of the specimen bag 300, i.e., from within body cavity “C”. The window 304 may extend along any or all of the length of the specimen bag 300 and may extend about any or all of the circumference of the specimen bag 300 and may be of any shape, such as circular or rectangular. In addition, the specimen bag 300 may include two or more windows (not shown) to permit viewing of the contents of the specimen bag 300 from various angles.

With additional reference to FIG. 3A, instead of, or in addition to, the window 304 in the wall of the specimen bag 300, the specimen bag 300 may include one or more viewing ports or optical connectors 306. In one embodiment, the viewing port 306 includes a base 308 that is fixedly secured to the specimen bag 300 and a flexible flange 310 for selectively receiving or engaging a distal end of the scope “S”. A sleeve 312 extends from the flange 310 of the viewing port 306 into the cavity 303 of the specimen bag 300 and includes a transparent closed free end 312a. The sleeve 312 receives the scope “S” and permits viewing of the contents of the specimen bag 300. Alternatively, the viewing port 306 includes a transparent window 314 positioned at an inner end of the flange 310 and may lay flush with the wall of the specimen bag 300. The transparent window 314 permits viewing of at least some of the contents of the specimen bag 300 without the requirement of having the scope “S” extend into the cavity 303 of the specimen bag 300.

During a laparoscopic procedure, the specimen bag 300 is received and positioned within the body cavity “C” of a patient using known methods. The tissue to be removed from the patient, e.g., gallbladder “G”, is placed within the cavity 303 of the specimen bag 300 through the open end 300a of the specimen bag 300. The open end 300a is then retracted through an incision “I” of the patient “P” to provide the surgeon with access to the cavity 303 of the specimen bag 300. A morcellator “M” is then positioned within the cavity 303 of the specimen bag 300 through the open end 300a of the specimen bag 300 and the scope “S” is positioned adjacent the window 304 or is secured to the viewing port 306 of the specimen bag 300 to permit viewing within the specimen bag 300. Insufflation gas can be supplied to the cavity 303 of the specimen bag 300 as described above.

As the morcellator “M” is operated within the cavity 303 of the specimen bag 300, the contents of the cavity 303 are viewed by the scope “S” through window 304 and/or viewing port 306. The flexibility of the flange 310 of the viewing port 306 and/or the flexibility of the specimen bag 300 allows the scope “S” to be manipulated to optimize visualization within the cavity 303 of the specimen bag 300. Upon removal of the tissue from the cavity 303 of the specimen bag 300 during morcellation or upon visual determination by the surgeon that the tissue has been sufficiently reduced in size to permit removal of the specimen bag 300 through the incision “I”, the morcellator “M” is removed from with the cavity 303 and, if necessary, the scope “S” is separated from the viewing port 306. The specimen bag 300 is then removed through the incision “I”.

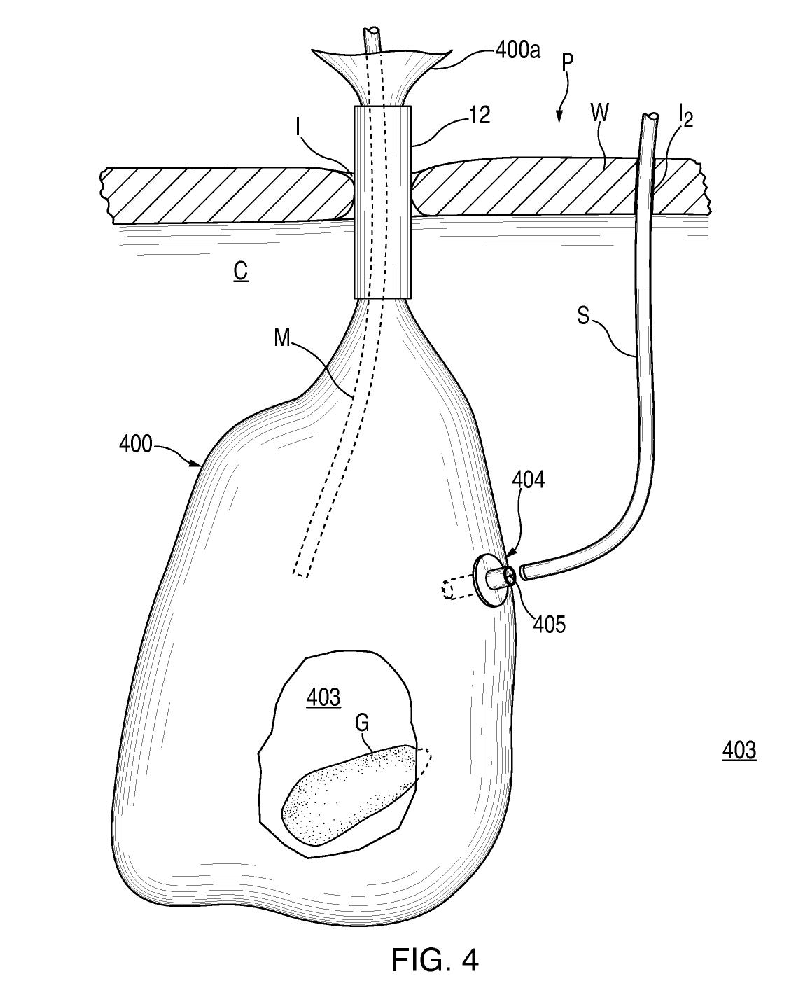

With reference now to FIG. 4, another embodiment of a specimen bag according to the present disclosure is shown generally as specimen bag 400. The specimen bag 400 includes an open end 400a and a closed end 400b and defines a cavity 403 for receiving tissue to be removed from the patient, e.g., gallbladder “G”. The specimen bag 400 further includes one or more ports 404 for at least receiving a scope “S” to permit viewing of the contents of the specimen bag 400.

The port 404 may be integrally formed with or securely affixed within a wall of the specimen bag 400. The port 404 defines at least one resealable or self-sealing opening 405 extending through the wall of the specimen bag 400 for receipt of the scope “S”. The port 404 is configured to seal opening 405 in the absence of the scope “S” and/or to receive the scope “S” in a fluid tight manner. The seal may be any conventional seal or valve, such as duckbill, joker, and zero-closure. Alternatively, the seal may include a combination of seals. The specimen bag 400 may include multiple ports (not shown) for accessing the specimen bag 400 from various angles.

During a laparoscopic procedure, the specimen bag 400 is received and positioned within the body cavity “C” of a patient using known methods. The tissue to be removed from the patient, e.g., gallbladder “G”, is placed within the cavity 403 of the specimen bag 400 through the open end 400a of the specimen bag 400. The open end 400a is then retracted through the incision “I” of the patient “P” to provide the surgeon with access to the cavity 403 of the specimen bag 400. A morcellator “M” is then received within the cavity 403 of the specimen bag 400 through the open end 400a of the specimen bag 400 and the scope “S” is received through the port 404 of the specimen bag 400 to permit viewing within the specimen bag 400. Insufflation gas can be supplied to the cavity 403 of the specimen bag 400 as described above.

As the morcellator “M” is operated within the cavity 403 of the specimen bag 400, the contents of the cavity 403 are viewed by the scope “S”. The port 404 is configured to permit manipulation of the scope “S” therethrough to permit visualization and/or optimize the view within the cavity 403 of the specimen bag 400. Upon removal of the tissue from the cavity 403 of the specimen bag 400 during morcellation or upon visual determination by the surgeon that the tissue has been sufficiently reduced in size to permit removal of the specimen bag 400 through the incision “I”, the morcellator “M” is removed from within the cavity 403 and the scope “S” is removed from within the viewing port 404. As noted above, the port 404 is configured to seal upon removal of the scope “S” from within opening 405. The specimen bag 400 is then removed directly through the incision “I”.

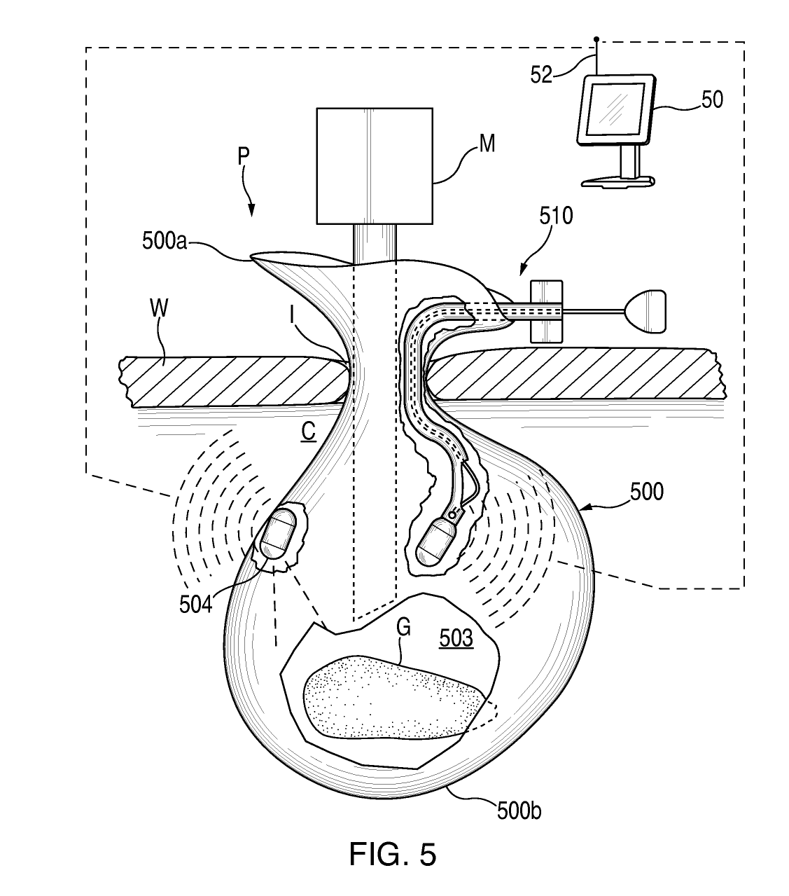

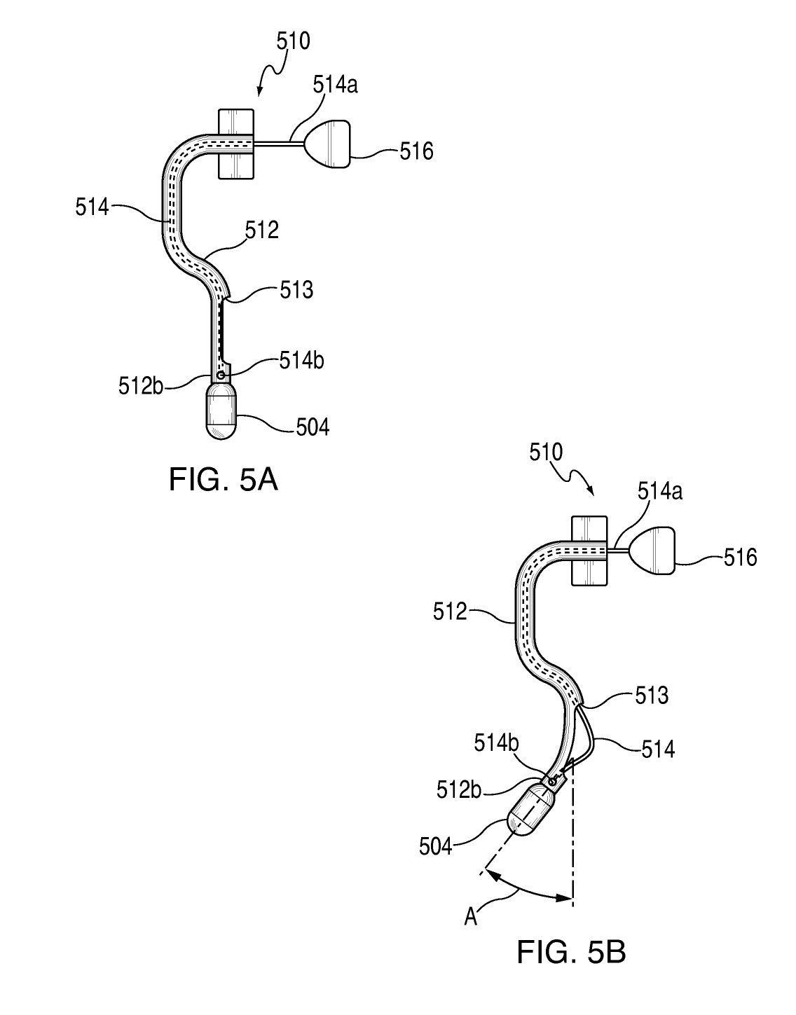

With reference now to FIG. 5, another embodiment of a specimen bag according to the present disclosure is shown generally as specimen bag 500. The specimen bag 500 includes an open end 500a and a closed end 500b and defines a cavity 503 for receiving tissue to be removed from the patient, e.g., gallbladder “G”. The specimen bag 500 includes one or more cameras 504 for providing a view of the contents of the specimen bag 500. The camera(s) 504 received within specimen bag 500 is wirelessly connected to a receiver 52 by Wi-Fi or through other wireless means for viewing of the contents of the specimen bag 500 on a monitor 50.

In embodiments, the camera 504 received within the specimen bag 500 is a pill camera. One or more pill cameras 504 may be secured to an interior wall of the specimen bag 500 to provide a view of the cavity 503 of the specimen bag 500. Alternatively the pill camera 504 is freely received within the specimen bag and includes a mechanism (not shown) for positioning the pill camera by remote control to optimize viewing of the contents within the cavity 503 of the specimen bag 500.

With additional reference to FIGS. 5A and 5B, in an alternative embodiment, camera 504 is attached to a steering mechanism 510 for positioning the camera 504 within the cavity 503 of the specimen bag 500 for optimal viewing. The steering mechanism 510, which is exemplary and not intended to limit the scope of the invention, includes a sleeve 512 and a rod 514 which is at least partially flexible and is received through the sleeve 512. The camera 504 is disposed on a distal end 512b of the sleeve 512. In embodiments, the sleeve 512 is formed of a flexible, pliable or shape memory material. A handle 516 is attached to a proximal end 514a of the flexible rod 514 for operable engagement by a surgeon. A distal end 514b of the flexible rod 514 is affixed to the distal end 512b of the sleeve 512 such that longitudinal advancement of the flexible rod 514 within the sleeve 514 causes the distal end of the sleeve 512 to articulate, as indicated by arrows “A”. The sleeve 512 defines a slot 513 to accommodate the flexible rod 514 during longitudinal advancement of the flexible rod 514. Articulation of the distal end 512b of the sleeve 512 allows the camera 504 to be selectively positioned for viewing within the cavity 503 of the specimen bag 500.

During a laparoscopic procedure, the specimen bag 500 is received and positioned within the body cavity “C” of a patient using known methods. When utilizing the pill camera 504 that is attached to the specimen bag 500, the pill camera 504 may be attached to the internal wall of the specimen bag prior to receiving the specimen bag 500 with the body cavity of the patient. The tissue to be removed from the patient, e.g., gallbladder “G”, is then placed within the cavity 503 of the specimen bag 500 through the open end 500a of the specimen bag 500. The open end 500a is next retracted through incision “I” of the patient “P” to provide the surgeon with access to the cavity 503 of the specimen bag 500.

If not already attached to or received within the specimen bag 500, the pill camera 504 is received within the specimen bag 500 through the open end 500a of the specimen bag 500. When utilizing the camera 504 attached to the steering mechanism 510, the camera 504 and the sleeve 512 of the steering mechanism 510 are inserted into the cavity 503 through the open end 500a of the specimen bag 500. A morcellator “M” is then received within the cavity 503 of the specimen bag 500 through the open end 500a of the specimen bag 500. Insufflation gas can be supplied to the cavity 503 of the specimen bag 500 as described above.

As the morcellator “M” is operated within the cavity 503 of the specimen bag 500, the camera 504 is positioned to view the contents of the cavity 503 of the specimen bag 500. As described above, the camera 504 is positioned for optimal viewing of the cavity 503 of the specimen bag 500 by remote control or by using the steering mechanism 510. As the tissue is morcellated, the camera 504 wirelessly communicates with the receiver 52 to provide an image to the monitor 50 for viewing by the surgeon.

Upon removal of the tissue from the cavity 503 of the specimen bag 500 during morcellation or upon visual determination by the surgeon that the tissue has been sufficiently reduced in size to permit removal of the specimen bag 500 through the incision “I”, the morcellator “M” is removed from within the cavity 503 of the specimen bag 500. The camera 504 may also be removed from the cavity 503 of the specimen bag 500. The specimen bag 500 and the pill camera 504 are then removed through the incision “I”.

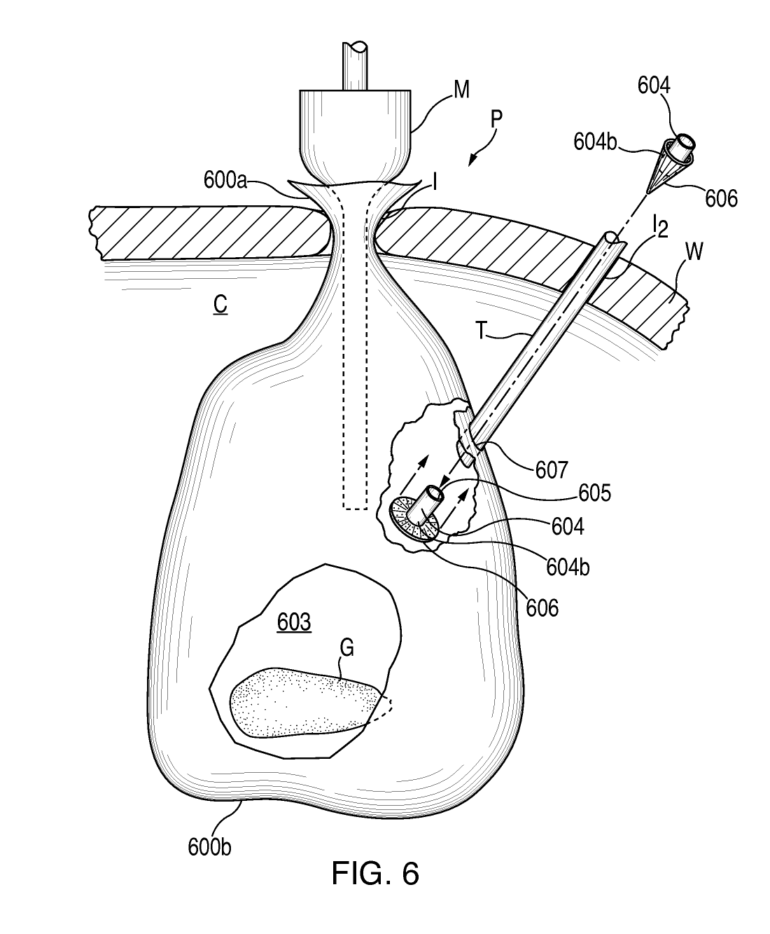

With reference now to FIG. 6, another embodiment according to the present disclosure is shown generally as specimen bag 600. The specimen bag 600 includes an open end 600a and a closed end 600b and defines a cavity 603 for receiving tissue to be removed from the patient “P”, e.g., gallbladder “G”. An appendage 604 is securely affixed to the specimen bag 600 during a procedure as described below. Although shown including only one appendage, it is envisioned that two or more appendages may be affixed to the specimen bag 600. The appendage 604 defines a channel 605 communicating with and providing access to the cavity 603 of the specimen bag 600. The channel 605 of the appendage 604 is dimensioned to selectively receive a morcellator “M”, a scope (not shown), and/or other instrument (not shown) in a fluid tight manner.

With continued reference to FIG. 6, the appendage 604 includes a flange 606 received about a distal end 604b thereof. The flange 606 collapses to permit insertion of the distal end 604b of the appendage 604 through an opening 607 formed in the wall of the specimen bag 600. The opening 607 may be formed with a sharpened trocar “T”, as shown, or with a scalpel or other cutting instrument. The sharpened trocar “T” may also be used to facilitate insertion of the distal end 604b of the appendage 604 through the opening 607 formed in the specimen bag 600. Once the distal end 604b of the appendage 604 is received through the opening 607 and within the cavity 603 of the specimen bag 600, the flange 606 is expanded and the appendage 604 is retracted through the opening 607 until the flange 606 engages the inner wall of the specimen bag 600. An adhesive on the flange 606 secures the flange 606 to the specimen bag 600 in a fluid tight manner. In an alternative embodiment, the flange 606 of the appendage 604 is secured to an external wall of the specimen bag 600. A proximal end of the appendage 604 is configured to be received through the abdominal wall “W” of the patient “P” to provide an additional opening for accessing the cavity 603 of the specimen bag 600.

During a laparoscopic procedure, the specimen bag 600 is received and positioned within the body cavity “C” of a patient using known methods. The tissue to be removed from the patient, e.g., gallbladder “G”, is placed within the cavity 603 of the specimen bag 600 through the open end 600a of the specimen bag 600. The open end 600a of the specimen bag 600 is then retracted through incision “I” of the patient “P” to provide the surgeon with access to the cavity 603 of the specimen bag 600.

One or more appendages 604 may then be attached to the specimen bag 600 as described above. An anchor or other holding means or mechanism (not shown) can be attached to the appendage 604 to prevent the appendage 604 from being retracted back through the incision “I₂” within the body cavity “C”.

The scope “S” is then inserted through the channels 605 of the appendages 604 and guided into the cavity 603 of the specimen bag 600 to permit viewing of the contents of the specimen bag 600 and the morcellator “M” is received through the open end 600a of the specimen bag 600. Insufflation gas can be provided to the cavity 603 of the specimen bag 600 as described above.

The positioning of the scope within the cavity 603 of the specimen bag 600 permits a surgeon to view the contents of the cavity 603 of the specimen bag 600 during operation of the morcellator “M”. Upon removal of the tissue from the cavity 603 of the specimen bag 600 during morcellation or upon determination that the tissue has been sufficiently morcellated to permit removal of the specimen bag 600 through the incision “I”, the scope is removed from channel 605 of the appendage 604 and the morcellator “M” is withdrawn from the cavity 603 of the specimen bag 600. The channel 605 of the appendage 604 is then sealed, as described above, to prevent leakage of any material from within the cavity 603 of the specimen bag 600 during removal of the specimen bag 600 through incision “I”. The specimen bag 600 is then removed from the body cavity “C” through the incision “I”. Alternatively, graspers may be used to grasp the open end of the appendage 604 and the appendage 604 is used to withdraw the specimen bag 600 from within the body cavity “C”.

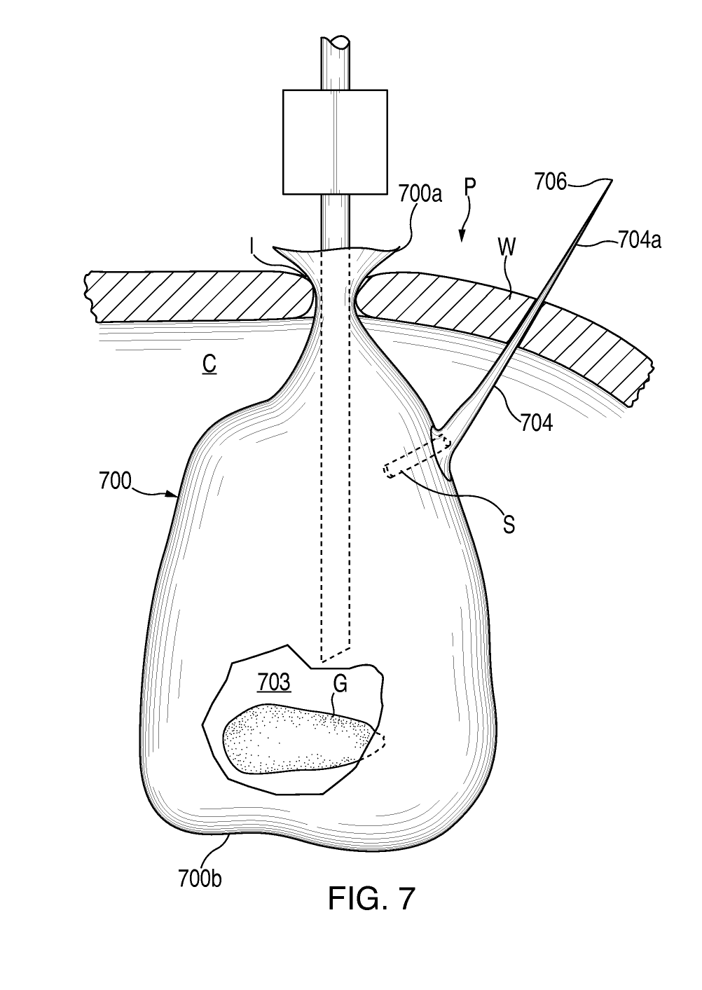

With reference now to FIG. 7, another embodiment of a specimen bag according to the present disclosure is shown generally as specimen bag 700. The specimen bag 700 includes an open end 700a and a closed end 700b and defines a cavity 703 for receiving tissue to be removed from the patient, e.g., gallbladder “G”. An appendage 704 is integrally formed with or securely affixed to the specimen bag 700. Although shown including only one appendage, it is envisioned that the specimen bag 700 may include a plurality of appendages. The appendage 704 defines a channel 705 communicating with and providing access to the cavity 703 of the specimen bag 700. The channel 705 of the appendage 704 is dimensioned to selectively receive a morcellator “M”, a scope “S”, and/or other instrument (not shown) in a fluid tight manner.

The appendage 704 includes a sealed end 704a forming a pointed tip or sharp edge 706 for penetrating through the abdominal wall “W” of the patient “P” when the specimen bag 700 is received with the body cavity “C”. The appendage 704 includes a length that, once received through the abdominal wall “W”, provides a surgeon sufficient material to detach the pointed tip 706 and direct the scope “S” and/or other instrument (not shown) therethrough. The pointed tip 706 may be formed of the same material as the specimen bag 700 or of a hard polymer, metal, or other suitable material.

Following the morcellation of the contents of the specimen bag 700 and removal of the scope “S” from within the channel 705 of the appendage 704, the channel 705 is sealed as described above, to prevent any material remaining in the cavity 703 of the specimen bag 700 from leaking into the body cavity “C” as the specimen bag 700 is removed from the patient “P”.

During a laparoscopic procedure, the specimen bag 700 is received and positioned within the body cavity “C” of a patient using known methods. The tissue to be removed from the patient “P”, e.g., gallbladder “G”, is placed within the cavity 703 of the specimen bag 700 through the open end 700a of the specimen bag 700. The open end 700a of the specimen bag 700 is then retracted through incision “I” of the patient “P” to provide the surgeon with access to the cavity 703 of the specimen bag 700.

At any time during the procedure, the surgeon may use forceps, graspers, and/or another instrument (not shown) to grasp the appendage 704 and direct the pointed end 706 of the appendage 704 through the abdominal wall “W” of the patient “P”. The sealed end 704a of the appendage 704 is then detached to provide access to the channels 705 of the appendage 704. An anchor or other holding means (not shown) may be attached to the appendage 704 to prevent the appendage 704 from being retracted through the abdominal wall “W” and into the body cavity “C”.

The morcellator “M” is then received through the open end 700a of the specimen bag 700, the scope “S” is inserted through the channel 705 the appendage 704, and each of the morcellator “M” and the scope “S” are guided into the cavity 703 of the specimen bag 700 to permit viewing and morcellation of the contents of the specimen bag 700. Insufflation gas can be supplied to the cavity 703 of the specimen bag 700 as described above.

The positioning of the scope “S” within the cavity 703 of the specimen bag 700 permits a surgeon to visualize the contents of the cavity 703 of specimen bag 700 during operation of the morcellator “M”. Upon removal of the tissue from the cavity 703 of the specimen bag 700 during morcellation or upon visual determination by the surgeon that the tissue has been sufficiently morcellated to permit removal of the specimen bag 700 through the incision “I”, the scope “S” and the morcellator “M” are withdrawn from the cavity 703 of the specimen bag 700. The channel 705 of the appendage 704 is then sealed to prevent leakage of any material from within the cavity 703 of the specimen bag 700 in the body cavity “C”. As described above, the channel 705 of the appendage 704 may be sealed by welding or by folding the end of the appendage 704 and suturing, stapling or otherwise securing the folded end of the appendage 704. The specimen bag 700 is then removed from the body cavity “C” through the incision “I”. Alternatively, graspers may be used to grasp the open end 700a of the appendage 704 and the appendage 704 is used to withdraw the specimen bag 700 from within the patient “P”.

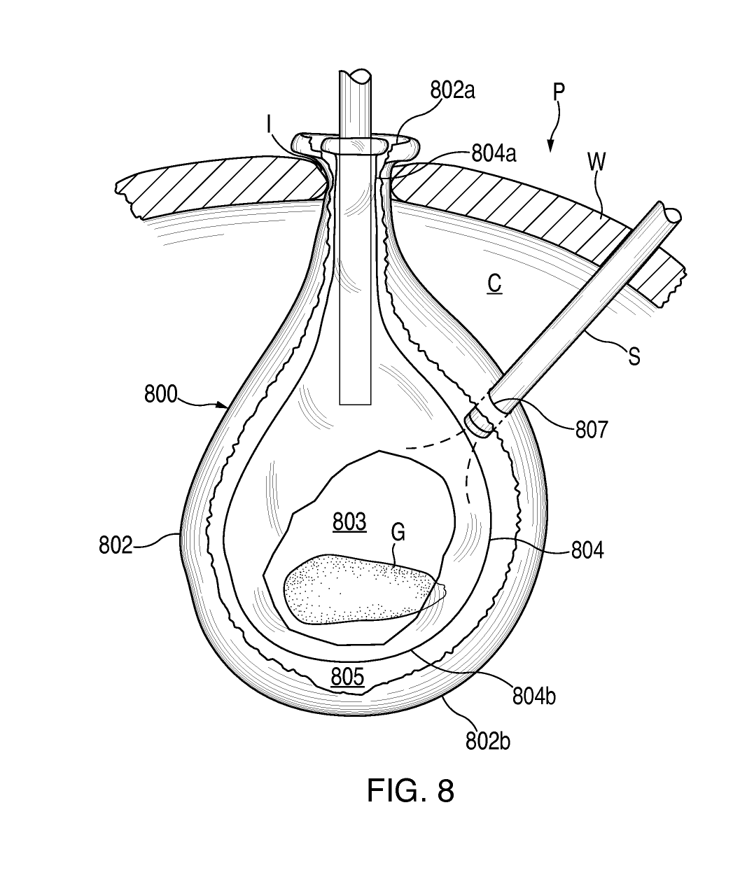

With reference now to FIG. 8, another embodiment of a specimen bag according to the present disclosure is shown generally as specimen bag 800. The specimen bag 800 includes an outer bag 802 and an inner bag 804. The outer bag 802 includes open end 802a and a closed end 802b and defines a cavity 803 in which inner bag 804 is received. The inner bag 804 includes an open end 804a and a closed end 804b and defines a cavity 805 for receiving tissue to be removed from the patient “P”, e.g., gallbladder “G”. The outer bag 802 is formed of rip stop nylon or other suitable material and the inner bag 804 is formed of a clear polymer or other transparent material. The outer bag 802 is of a sufficient size that when the outer and inner bags 802, 804 are insufflated there is room between the inner bag 804 and the outer bag 802 to permit manipulation of a scope “S” received through an opening 807 formed in the outer bag 802.

During a laparoscopic procedure, the specimen bag 800, including the outer and inner bags 802, 804, is received and positioned within the body cavity “C” of a patient “P” using known methods. The tissue to be removed from the patient, e.g., gallbladder “G”, is then placed within the cavity 805 of the inner bag 804 through the open end 804a of the inner bag 804. The open ends 802a, 804a of the outer and inner bags 802, 804, respectively, of the specimen bag 800 are then retracted through the incision “I” of the patient “P” to provide the surgeon with access to the cavities 803, 805 of the outer and inner bags 802, 804, respectively, of the specimen bag 800.

Insufflation gas is then provided to the cavity 803 of the outer bag 802 through open end 802a to inflate the outer bag 802 thereby providing space between the outer bag 802 and the inner bag 804. An opening 807 is then made in the outer bag 802 using a scalpel, cutting trocar, or other suitable instrument to provide access for receipt of the scope “S”. The scope “S” is then inserted through the opening 807 in the outer bag 802. A morcellator “M” is next received within the cavity 805 of the inner bag 804 through the open end 804a of the inner bag 804.

Insufflation gas may continue to be provided to the cavity 803 of the outer bag 802 through the open end 802a or the insufflation gas may be provided through the opening 807 in the outer bag 802 or through an additional opening formed in the outer bag 802. The morcellator “M” may provide insufflation gas to the cavity 805 of the inner bag 804. Alternatively, insufflation gas is provided to the cavity 805 of the inner bag 804 using an insufflation trocar (not shown) or other means. As the morcellator “M” is operated within the cavity 805 of the inner bag 804, the scope “S” is manipulated within the outer bag 804 to view the contents of the cavity 805 of the inner bag 804 through the inner bag 804.

Upon removal of the tissue from the cavity 805 of the inner bag 804 during morcellation or upon visual determination by the surgeon that the tissue has been sufficiently reduced in size, for example to permit removal of the specimen bag 800 through the incision “I”, the morcellator “M” is removed from within the cavity 805 of the inner bag 804 and the scope “S” is removed from within the cavity 803 of the outer bag 802. The specimen bag 800, including the outer and inner bags 802, 804 is then removed through the incision “I”.

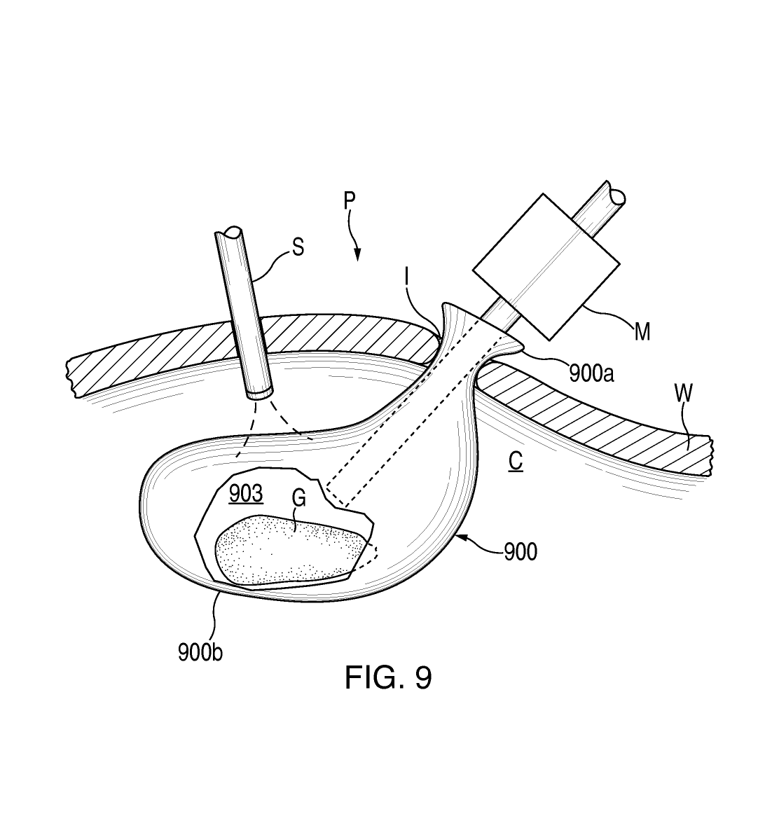

With reference now to FIG. 9, another embodiment of a specimen bag according to the present disclosure is shown generally as specimen bag 900. The specimen bag 900 includes an open end 900a and a closed end 900b and defines a cavity 903 for receiving tissue to be removed from the patient, e.g., gallbladder “G”. The specimen bag 900 is formed of a material that permits viewing therethrough using a thermal imaging scope “S”.

During a laparoscopic procedure, the specimen bag 900 is received and positioned within the body cavity “C” of a patient “P” using known methods. The tissue to be removed from the patient, e.g., gallbladder “G”, is placed within the cavity 903 of the specimen bag 900 through the open end 900a of the specimen bag 900. The open end 900a is then retracted through the incision “I” of the patient “P” to provide the surgeon with access to the cavity 903 of the specimen bag 900.

A morcellator “M” is received within the cavity 903 of specimen bag 900 through the open end 900a of the specimen bag 900. Insufflation gas is provided to the cavity 903 of the specimen bag 900 as described above. The thermal imaging scope “S” is then positioned within body cavity “C” to provide a thermal image of the contents of the specimen bag 900.

Upon removal of the tissue from the cavity 903 of the specimen bag 900 during morcellation or upon visual determination by the surgeon that the tissue has been sufficiently reduced in size to permit removal of the specimen bag 900 through the incision “I”, the morcellator “M” is removed from within the cavity 903 of the specimen bag 900. The specimen bag 900 is then removed through the incision “I”.

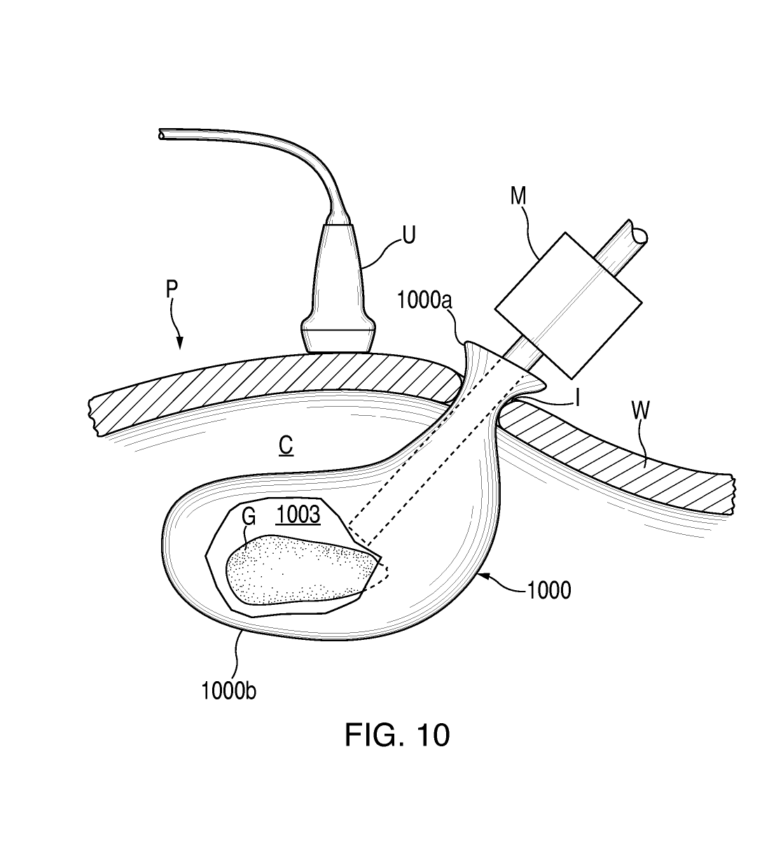

With reference now to FIG. 10, another embodiment of a specimen bag according to the present disclosure is shown generally as specimen bag 1000. The specimen bag 1000 includes an open end 1000a and a closed end 1000b and defines a cavity 1003 for receiving tissue to be removed from the patient, e.g., gallbladder “G”. The specimen bag 1000 is formed of a material that permits viewing therethrough using ultrasound.

During a laparoscopic procedure, the specimen bag 1000 is received and positioned within the body cavity “C” of a patient “P” using known methods. The tissue to be removed from the patient, e.g., gallbladder “G”, is placed within the cavity 1003 of the specimen bag 1000 through the open end 1000a of the specimen bag 1000. The open end 1000a is then retracted through an incision “I” of the patient “P” to provide the surgeon with access to the cavity 1003 of the specimen bag 1000.

A morcellator “M” is received within cavity 1003 of the specimen bag 1000 through the open end 1000a of the specimen bag 1000. Insufflation gas is provided to the cavity 1003 of the specimen bag 1000 as described above. An ultrasound transducer or wand “U” is then placed against the abdominal wall “W” proximate to the specimen bag 1000 to provide an image to the surgeon of the connects of the cavity 1003 of the specimen bag 1000. Thus, visualization of the contents of the specimen bag 1000 is accomplished without accessing the body cavity “C” of the patient “P” with a scope or other viewing means.

Upon removal of the tissue from the cavity 1003 of the specimen bag 1000 during morcellation or upon visual determination by the surgeon that the tissue has been sufficiently reduced in size to permit removal of the specimen bag 1000 through the incision “I”, the morcellator “M” is removed from within the cavity 1003 of the specimen bag 1000. The specimen bag 1000, including the morcellated tissue, if any, is then removed through the incision “I”.

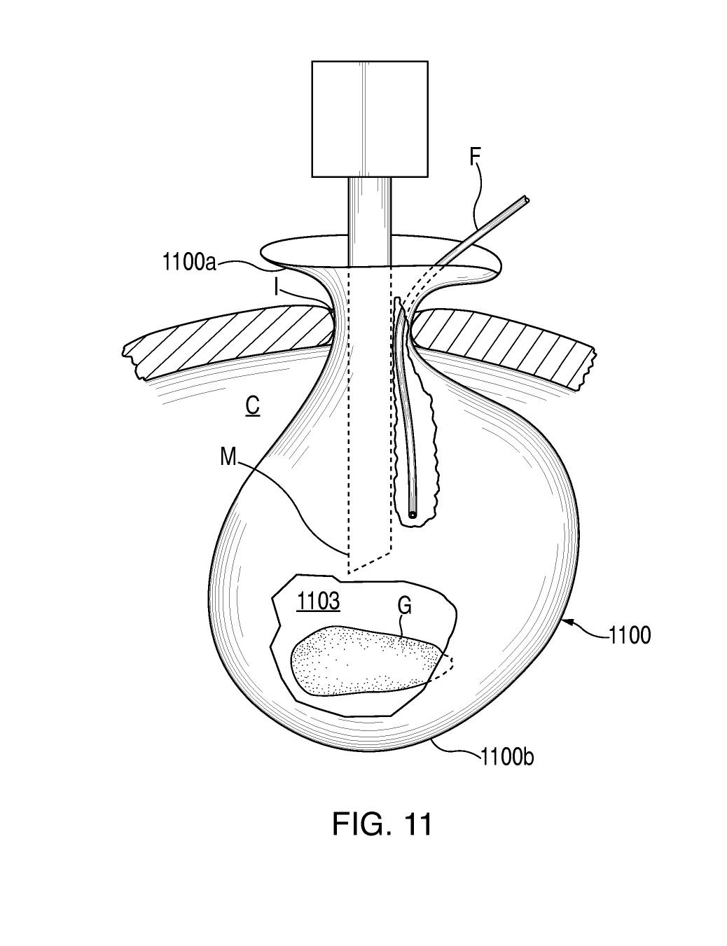

With reference now to FIG. 11, another embodiment of a specimen bag according to the present disclosure is shown generally as specimen bag 1100. The specimen bag 1100 includes an open end 1100a and a closed end 1100b and defines a cavity 1103 for receiving tissue to be removed from the patient, e.g., gallbladder “G”. The specimen bag 1100 includes a fiber optic “F” for providing a view of the contents of the specimen bag 1100. As will be described in further detail below, the fiber optic “F” is received through the open end 1100a of the specimen bag 1100, and therefore, does not require an addition opening through the abdominal wall “W” and/or through the specimen bag 1100. The size of the fiber optic “F” permits a wider range of viewing within the cavity 1103 of the specimen bag 1100.

During a laparoscopic procedure, the specimen bag 1100 is received and positioned within the body cavity “C” of a patient using known methods. The tissue to be removed from the patient, e.g., gallbladder “G”, is placed within the cavity 1103 of the specimen bag 1100 through the open end 1100a of the specimen bag 1100. The open end 1100a is then retracted through an incision “I” of the patient “P” to provide the surgeon with access to the cavity 1103 of the specimen bag 1100.

A morcellator “M” is then received within the cavity 1103 of the specimen bag 1100 through the open end 1100a of the specimen bag 1100. As noted above, the fiber optic “F” is also received through the open end 1100a of the specimen bag 1100. Insufflation gas is provided to the cavity 1103 of the specimen bag 1100 as described above. As the morcellator “M” is operated within the cavity 1103 of the specimen bag 1100, the fiber optic “F” is manipulated to view the contents of the cavity 1103 of the specimen bag 1100.

Upon removal of the tissue from the cavity 1103 of the specimen bag 1100 during morcellation or upon visual determination by the surgeon that the tissue has been sufficiently reduced in size to permit removal of the specimen bag 1100 through the incision “I”, the morcellator “M” is removed from with the cavity 1103 of the specimen bag 1100. The fiber optic “F” is also removed from the cavity 1103 of the specimen bag 1100. The specimen bag 1100 is then removed through the incision “I”.

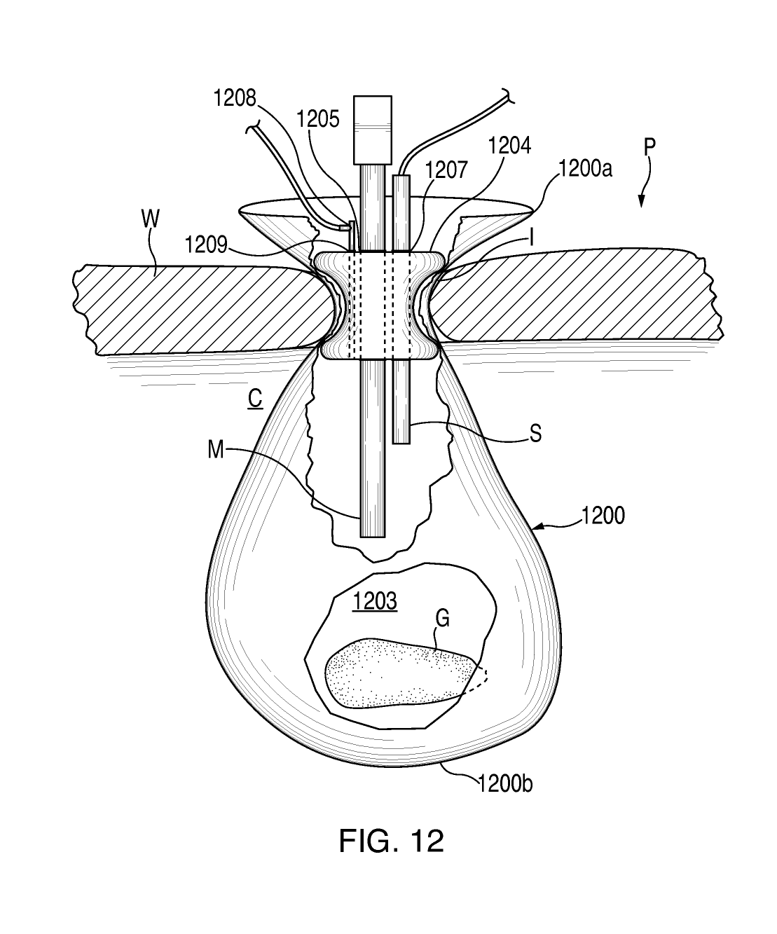

With reference now to FIG. 12, another embodiment of a specimen bag according to the present disclosure is shown generally as specimen bag 1200. The specimen bag 1200 includes an open end 1200a and a closed end 1200b and defines a cavity 1203 for receiving tissue to be removed from the patient, e.g., gallbladder “G”. The specimen bag 1200 includes a multi-lumen port 1204 received within the open end 1200a of the specimen bag 1200 subsequent to withdrawal of the open end 1200a of the specimen bag 1200 through the incision “I”. In embodiments, and as shown, the multi-lumen port 1204 includes a substantially hour-glass shape. As will be described in further detail below, the multi-lumen access port 1204 includes at least two openings and/or lumen 1205, 1207 for receiving at least a morcellator “M” and a scope “S”. Each of the lumen 1205, 1207 may include a seal or valve as described above. The multi-lumen access port 1204 may further include an insufflation valve 1208 in communication with an insufflation lumen 1209 for supplying insufflation gas to the cavity 1203 of the specimen bag 1200.

During a laparoscopic procedure, the specimen bag 1200 is received and positioned within the body cavity “C” of a patient using known methods. The tissue to be removed from the patient, e.g., gallbladder “G”, is placed within the cavity 1203 of the specimen bag 1200 through the open end 1200a of the specimen bag 1200. The open end 1200a is then retracted through the incision “I” of the patient “P” to provide the surgeon with access to the cavity 1203 of the specimen bag 1200.

The multi-lumen access port 1204 is then received within open end 1200a of specimen bag 1200 and within the incision “I” in the patient “P”. The multi-lumen access port 1204 creates a fluid tight seal within open end 1200a of the specimen bag 1200 and between the specimen bag 1200 and the abdominal wall “W”.

A morcellator “M” is then received within the cavity 1203 of the specimen bag 1200 through the lumen 1205 in the multi-lumen access port 1204 and the scope “S” is received within the cavity 1203 of the specimen bag 1200 through the lumen 1207 in the multi-lumen access port 1204. Insufflation gas can be provided to the cavity 1203 through insufflation port 1208 or as described above. As the morcellator “M” is operated within the cavity 1203 of the specimen bag 1200, the scope “S” is manipulated through multi-lumen port 1200 to view the contents of the cavity 1203 of the specimen bag 1200.

Upon removal of the tissue from the cavity 1203 of the specimen bag 1200 during morcellation or upon visual determination by the surgeon that the tissue has been sufficiently reduced in size to permit removal of the specimen bag 1200 through the incision “I”, the morcellator “M” and scope “S” are removed from with the cavity 1203 of the specimen bag 1200 through the multi-lumen access port 1204 and the multi-lumen access port 1204 is removed from within the open end 1200a of the specimen bag 1200. The specimen bag 1200 is then removed through the incision “I”. Alternatively, the specimen bag 1200 is removed through the incision “I” while the multi-lumen access port 1204 remains received within the open end 1200a of the specimen bag 1200.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

1. A specimen bag comprising:
a bag having an open end and a closed end and defining a cavity; and
an appendage extending from the bag and defining a channel in communication with the cavity of the bag.

2. The specimen bag of claim 1, wherein a proximal end of the appendage is sealed.

3. The specimen bag of claim 1, further including at least one additional appendage extending from the bag.

4. The specimen bag of claim 1, wherein the appendage is configured to receive a surgical instrument through the channel in a sealed manner.

5. The specimen bag of claim 1, wherein the appendage includes a seal member for sealing the channel in the absence of a surgical instrument received therethrough.

6. The specimen bag of claim 1, wherein the appendage includes a length sufficient to be received through an opening in tissue when the bag is disposed within a body cavity.

7. A method of viewing the contents of a specimen bag, the method comprising:
placing tissue within a cavity of a specimen bag through an open end of the specimen bag;
opening a proximal end of an appendage extending from the specimen bag; and
receiving an instrument through the appendage into the cavity of the specimen bag.

8. The method of claim 7, further including directing the proximal end of the appendage through an incision in tissue prior to opening the proximal end of the appendage.

9. The method of claim 7, further including sealing the appendage after removing the instrument from the appendage.

10. The method of claim 7, further including manipulating the tissue within the cavity.

11. The method of claim 9, further including receiving an open end of the specimen bag through an incision in tissue.

12. A specimen bag comprising:
a bag having an open end and a closed end and defining a cavity; and
a visualization member for viewing within the cavity of the bag.

13. The specimen bag of claim 12, wherein the visualization member includes a transparent window formed in the bag.

14. The specimen bag of claim 12, wherein the visualization member includes a viewing port secured to the bag.

15. The specimen bag of claim 12, further including at least one additional visualization member.

16. A method of viewing the contents of a specimen bag, the method comprising:
placing tissue within a cavity of a specimen bag including a visualization member; and
positioning a scope adjacent the visualization member of the specimen bag.

17. The method of claim 16, further including receiving an open end of the specimen bag through an opening in tissue.

18. A specimen bag comprising:
a bag having an open end and a closed end and defining a cavity; and
a port extending through a wall of the bag, the port defining a resealable opening for selectively receiving a surgical instrument.

19. The specimen bag of claim 18, further including at least one addition port extending through the wall of the bag.

20. A method of viewing the contents of a specimen bag, the method comprising:
placing tissue within a cavity of a specimen bag through an open end of the specimen bag; and
receiving a scope through a resealable opening in a port extending through a wall of the bag.
